(19) United States
(12) Patent Application Publication
(10) Pub. No.: US 2004/0244086 A1
(43) Pub. Date: Dec. 9, 2004

(54) Detachable adaptor for glasses

(76) Inventor: Kyung-Nam Kim, Daegu-city (KR)

Correspondence Address: SHLESINGER, ARKWRIGHT & GARVEY LLP, 3000 South Eads Street, Arlington, VA, 22202, US

(21) Appl. No.: 10/727,982

(22) Filed: Dec. 5, 2003

(30) Foreign Application Priority Data
May 15, 2003 (KR) 20-2003-0014971

(51) Int. Cl. A61F 9/00

(52) U.S. Cl. 2/13

(57) ABSTRACT

A detachable adaptor for glasses by which shelter glasses are detachably mounted immediately in front of main glasses. The main glasses include a main frame which holds a pair of main lenses, and the shelter glasses include a shelter frame which has magnetic lockers at both ends thereof and holds a pair of shelter lenses. The detachable adaptor has first and second clamp legs to clamp the adaptor to a desired position of the main frame. The first and second clamp legs are jointed at centers thereof to each other by a hinge. The detachable adaptor further has a magnet unit provided at a rear end of the first clamp leg and an adjusting screw provided at a rear end of the second clamp leg. The adjusting screw has a role as a spacer to adjust a clamping force of the first and second clamp legs when clamping the adaptor to the desired position of the main frame. Therefore, the detachable adaptor for glasses of the present invention is mounted to a desired position of the main glasses and selectively changes magnetic poles of a magnet unit provided therein, so that a variety of shelter glasses are selectively attached to the main frame of the main glasses by use of the detachable adaptor.

### CROSS-REFERENCE TO RELATED APPLICATION

This application claims priority of Korean application no. 20-2003-0014971, filed May 15, 2003, which is incorporated herein by reference.

### BACKGROUND OF THE INVENTION

1. Field of the Invention

The present invention relates generally to adaptors for glasses, and more particularly, to a detachable adaptor for glasses which is mounted to a desired position of main glasses and selectively changes magnetic poles of a magnet unit provided therein, so that a variety of shelter glasses are selectively attached to the main frame of the main glasses by use of the detachable adaptor.

2. Description of the Related Art

Generally, glasses are classified into three types, which are main glasses to improve sight, such as short sightedness, far sightedness, astigmatism, presbyopia, and etc., shelter glasses to protect eyes from intensive light, harmful radiation, such as ultraviolet rays and infrared rays, dusts, and a mechanical permeation, and fashionable glasses for fashion.

A variety of the shelter glasses, such as sunglasses to intercept the ultraviolet rays, and safety goggles have been used in a variety of fields, such as a dental clinic, fishing, and a welding, have been widely used. The requirements for the shelter glasses have rapidly increased.

However, the conventional shelter glasses are disadvantageous as follows. When a user, who has poor sight and wears main glasses, requires shelter glasses, the user must use specific shelter glasses which are made by coating a shielding material and a coloring material on the lenses of the main glasses. Therefore, the conventional shelter glasses impose additional expenses on the user, because the user must give an order for the specific shelter glasses.

To solve the above-mentioned problems, a magnetic shelter frame for glasses has been proposed. As shown in FIG. 1, specific main glasses used with conventional shelter glasses having the magnetic shelter frame include a main frame 2 which holds a pair of main lenses 1 and has a pair of magnet units 3 at predetermined portions of the main frame 2 to generate magnetic forces. The shelter glasses used with the specific main glasses include a pair of shelter lenses 4 to protect the eyes of a user and the magnetic shelter frame 5. The magnetic shelter frame 5 has a pair of magnetic lockers 51 which are provided at both ends of the shelter frame 5 to be respectively attached to the pair of magnet units 3 of the main glasses by the magnetic forces generated by the magnet units 3.

In the meantime, detachable-type shelter glasses were proposed by the inventor of the present invention in Korean Patent Application NO. 2002-32128 (Filed: July. 8, 2002), entitled ‘DETACHABLE SHELTER GLASSES USING MAGNETS’. The above-mentioned detachable-type shelter glasses are primarily attached to specific main glasses by connecting units, and are secondarily attached to the main glasses by the magnetic forces of permanent magnets of the main glasses, so that the shelter glasses are simply and stably attached to the main glasses.

However, the conventional magnetic shelter frame for glasses and the conventional detachable-type shelter glasses have problems in that they are used with only the main glasses having the magnet units to magnetically attach the shelter glasses having the magnetic lockers. Furthermore, even though the conventional shelter glasses are used with main glasses having the magnet units, the position and magnetic poles of the magnet units of the main glasses must respectively correspond to those of the magnetic lockers of the shelter glasses, so that the compatibility of the conventional shelter glasses is reduced.

Therefore, the conventional shelter glasses impose additional expenses on the user, because the shelter glasses are used with only specific main glasses having the magnet units which are respectively correspond to the positions and magnetic poles of the magnetic lockers of the shelter glasses.

### SUMMARY OF THE INVENTION

Accordingly, the present invention has been made keeping in mind the above problems occurring in the prior art, and an object of the present invention is to provide a detachable adaptor for glasses which is mounted to a desired position of main glasses and selectively changes magnetic poles of a magnet unit provided therein, so that a variety of shelter glasses are selectively attached to the main frame of the main glasses by use of the detachable adaptor.

Additional aspects and/or advantages of the invention will be set forth in part in the description which follows and, in part, will be obvious from the description, or may be learned by practice of the invention.

The above and/or other aspects are achieved by providing a detachable adaptor for glasses by which shelter glasses are detachably mounted immediately in front of main glasses. The main glasses include a main frame which holds a pair of main lenses, and the shelter glasses include a shelter frame which has magnetic lockers at both ends thereof and holds a pair of shelter lenses. The detachable adaptor includes first and second clamp legs to clamp the adaptor to a desired position of the main frame. The first and second clamp legs are jointed at centers thereof to each other by a hinge. The detachable adaptor further includes a magnet unit provided at a rear end of the first clamp leg, and an adjusting screw provided at a rear end of the second clamp leg. The adjusting screw has a role as a spacer to adjust a clamping force of the first and second clamp legs when clamping the adaptor to the desired position of the main frame.

The rear end of the second clamp leg may have an internal thread to engage with the adjusting screw, and the rear end of the first clamp leg may have a flat surface to be in contact with an inside end of the adjusting screw, so that the first and second clamp legs are opened around the hinge at the rear ends thereof and are closed around the hinge at front ends thereof when the adjusting screw rotates in a predetermined direction, thus clamping the adapter to the desired position of the main frame.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of conventional specific main glasses and shelter glasses;

FIG. 2 is a perspective view of main glasses and shelter glasses having a detachable adaptor, according to an embodiment of the present invention;

FIG. 3 is a top view showing clamping positions of a plurality of detachable adaptors of the present invention, when the detachable adaptors are clamped to desired positions of a main frame of the main glasses; and

FIGS. 4A, 4B, 4C and 4D are views respectively showing different states of the detachable adaptor of the present invention, when the detachable adaptor is mounted to the main frame of the main glasses.

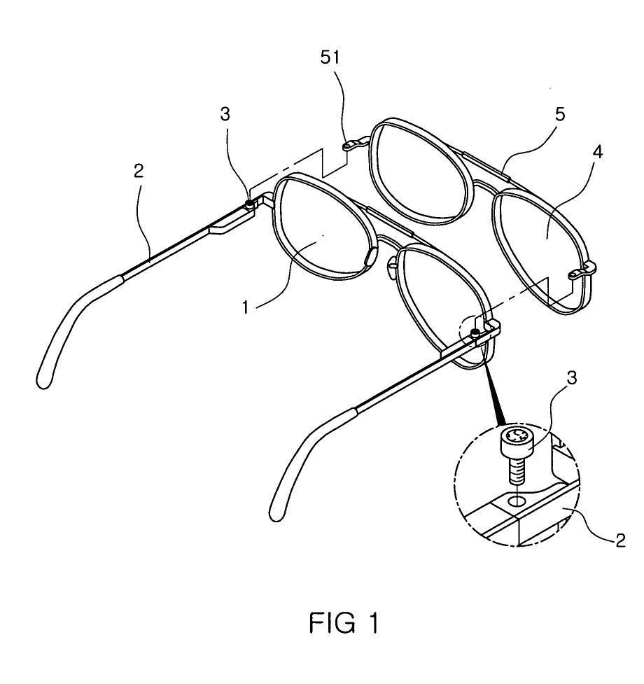

### DESCRIPTION OF THE PREFERRED EMBODIMENTS

Hereinafter, embodiments of the present invention will be described in detail with reference to the attached drawings.

Reference now should be made to the drawings, in which the same reference numerals are used throughout the different drawings to designate the same or similar components.

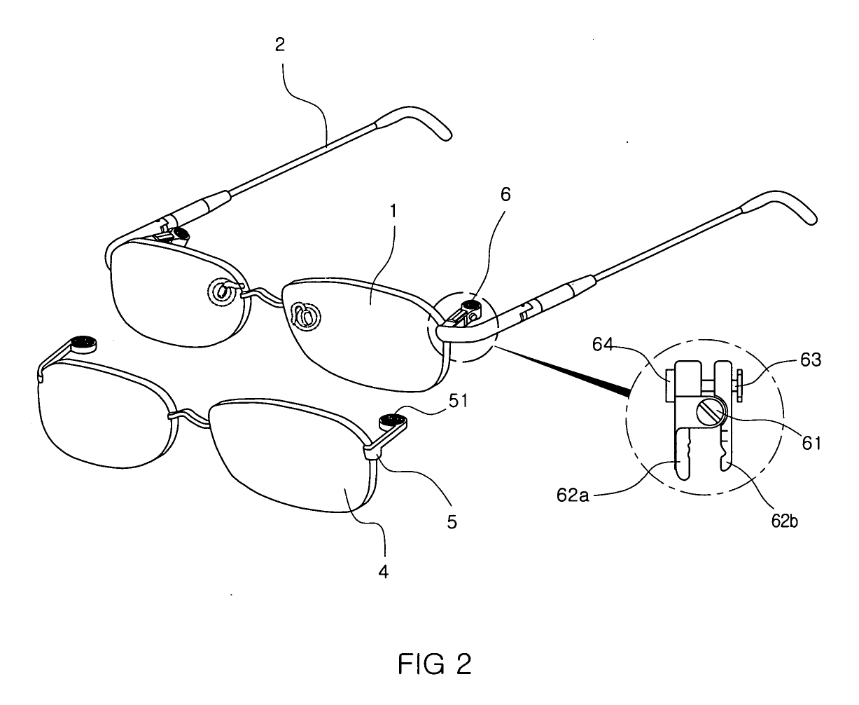

As shown in FIG. 2, a plurality of detachable adaptors 6 of the present invention are mounted to a desired positions of a main frame 2 of main glasses, such that shelter glasses are detachably mounted immediately in front of the main glasses 1 by the detachable adaptors 6. The main glasses include the main frame 2 which holds a pair of main lenses 1. The shelter glasses include a shelter frame 5 which has two magnetic lockers 51 at both ends thereof and holds a pair of shelter lenses 4 therein. Each of the detachable adaptors 6 has first and second clamp legs 62a and 62b to clamp the detachable adaptor 6 to a desired position of the main frame 2. At this time, the first and second clamp legs 62a and 62b are jointed at centers thereof to each other by a hinge 61. Each of the detachable adaptors 6 further has a magnet unit 64 which is provided at a rear end of the first clamp leg 62a, and an adjusting screw 63 which is provided at a rear end of the second clamp leg 62b. The adjusting screw 63 has a role as a spacer to adjust a clamping force of the first and second clamp legs 62a and 62b when clamping the detachable adaptor 6 to the desired position of the main frame 2 of the main glasses.

Next, the operation of the detachable adaptor 6 of the present invention will be described herein below.

In case that a user requires to wear the shelter glasses while the user wears the main glasses, the shelter glasses are primarily placed in front of the main glasses. Thereafter, the user attaches the shelter glasses to the main glasses while the magnetic lockers 51 of the shelter glasses are respectively placed on the magnet units 64 of the detachable adaptors 6.

In case that the shelter glasses are required to be removed from the main glasses, the user removes the shelter glasses from the main glasses by detaching the magnetic lockers 51 of the shelter glasses from the magnet units 64 of the detachable adaptors 6.

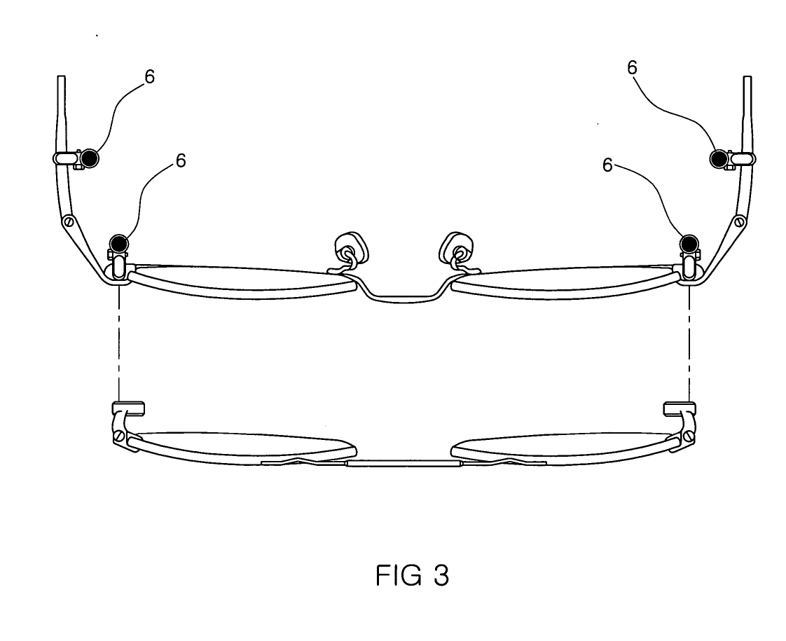

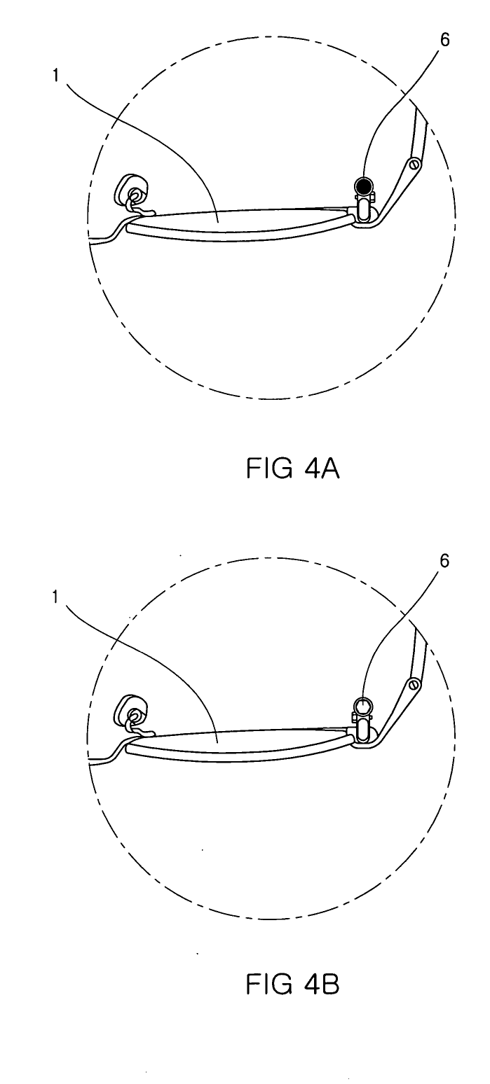

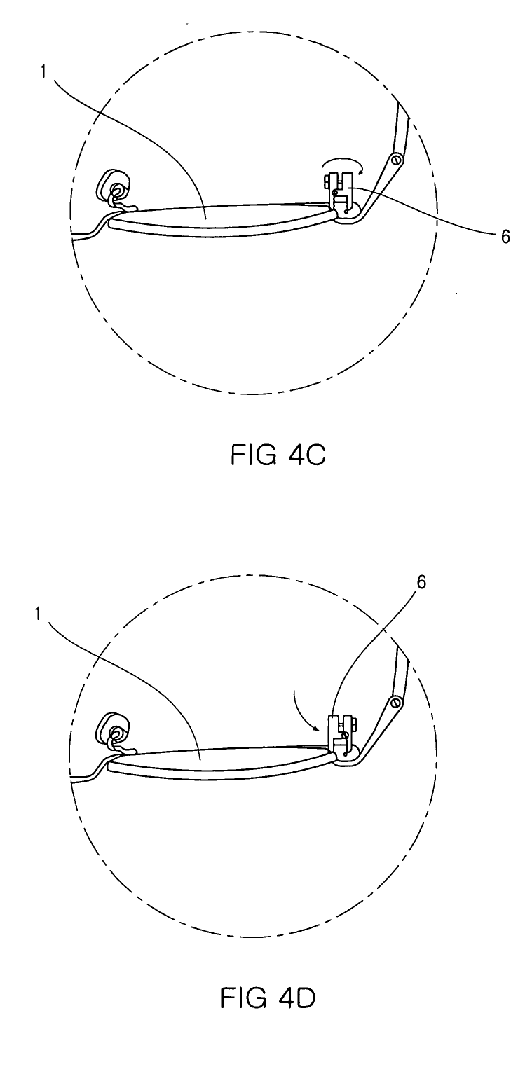

As shown in FIG. 3, the detachable adaptors 6 may be attached to any positions of the main frame 2 of the main glasses. In addition, the magnet unit 64 of each of the detachable adaptors 6 may be pointed in any direction, that is, upward, downward, leftward, and rightward, while the detachable adaptors 6 are attached to the desired positions of the main frame 2 of the main glasses.

When the detachable adaptors 6 are required to be mounted to the main frame 2 of the main glasses, each of the detachable adaptors 6 is placed on a desired position of the main frame 2 of the main glasses, so that the first and second clamp legs 62a and 62b of the detachable adaptor 6 clamp the detachable adaptor 6 to the main frame 2. At this time, the rear end of the second clamp leg 62b has an internal thread to engage with the adjusting screw 63, while the rear end of the first clamp leg 62a has a flat surface to be in contact with an inside end of the adjusting screw 63. Accordingly, the first and second clamp legs 62a and 62b are opened around the hinge 61 at the rear ends thereof and are closed around the hinge 61 at front ends thereof when the adjusting screw 63 rotates in a predetermined direction. The detachable adapter 6 is thus stably and firmly clamped to the desired position of the main frame 2.

As described above, the present invention provides a detachable adaptor for glasses which is mounted to a desired position of main glasses and selectively changes magnetic poles of a magnet unit provided therein, so that a variety of shelter glasses are selectively attached to the main frame of the main glasses by use of the detachable adaptor.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A detachable adaptor for glasses by which shelter glasses are detachably mounted immediately in front of main glasses, the main glasses including a main frame which holds a pair of main lenses, and the shelter glasses including a shelter frame which has magnetic lockers at both ends thereof and holds a pair of shelter lenses, the detachable adaptor comprising:
first and second clamp legs to clamp the adaptor to a desired position of the main frame, the first and second clamp legs being jointed at centers thereof to each other by a hinge;
a magnet unit provided at a rear end of the first clamp leg; and
an adjusting screw provided at a rear end of the second clamp leg, the adjusting screw having a role as a spacer to adjust a clamping force of the first and second clamp legs when clamping the adaptor to the desired position of the main frame.

2. The detachable adaptor according to claim 1, wherein the rear end of the second clamp leg has an internal thread to engage with the adjusting screw, and the rear end of the first clamp leg has a flat surface to be in contact with an inside end of the adjusting screw, so that the first and second clamp legs are opened around the hinge at the rear ends thereof and are closed around the hinge at front ends thereof when the adjusting screw rotates in a predetermined direction, thus clamping the adapter to the desired position of the main frame.

3. A device, comprising:
a) a main glasses including a main frame holding a pair of main lenses;
b) a shelter glasses including a shelter frame holding a pair of shelter lenses, the shelter frame holding the pair of shelter lenses substantially directly adjacent the main lenses;
c) the shelter glasses being detachably mounted in front of the main glasses, the shelter glasses including magnetic locking elements at both ends;
d) a detachable adapter configured for detachably attaching the shelter glasses in front of the main glasses, the detachable adapter including:
i) first and second clamp legs configured for clamping the adapter in a desired position relative to the main frame, the first and second clamp legs being hingedly attached to each other;
ii) a magnet unit provided at a rear portion of the first clamp leg; and
iii) an adjusting element provided at a rear portion of the second clamp leg, the adjusting element being configured for adjusting a clamping force of the first and second clamp legs when clamping the adapter in the desired position on the main frame.

4. The device as in claim 3, wherein:
a) the rear portion of the second clamp leg is configured for engaging with the adjusting element; and
b) a rear portion of the first clamp leg includes a flat surface configured for contacting an inside portion of the adjusting element, so that the first and second clamp legs open at the rear ends thereof and close at the front ends thereof when the adjusting element is adjusted, thereby clamping the adapter in the desired position on the main frame.
